(19) United States
(12) Patent Application Publication
(10) Pub. No.: US 2024/0408266 A1
(43) Pub. Date: Dec. 12, 2024

(54) WOUND DRESSING, WOUND GEL AND COSMETIC

(71) Applicant: PolyMedics Innovations GmbH, Kirchheim unter Teck (DE)

(72) Inventors: Heinrich Planck, Nürtingen (DE); Erhard Müller, Stuttgart (DE); Svenja Reimer, Aichtal (DE); Christian Planck, Kircheim (DE); Peter Erken, Tübingen (DE)

(73) Assignee: PolyMedics Innovations GmbH, Kirchheim unter Teck (DE)

(21) Appl. No.: 18/748,428

(22) Filed: Jun. 20, 2024

Related U.S. Application Data
(63) Continuation of application No. PCT/EP2022/087354, filed on Dec. 21, 2022.

(30) Foreign Application Priority Data
Dec. 23, 2021 (DE) 10 2021 215 013.6

(51) Int. Cl. A61L 15/24

(52) U.S. Cl. CPC A61L 15/24

(57) ABSTRACT

A synthetic wound agent is in the form of a bulk material with particles with a maximum dimension (d) of 5 millimeters. The particles each have a porosity of over 80% and each include a co-and/or terpolymer based on polyhydroxycarboxylic acids, namely the monomers lactide, trimethylene carbonate, caprolactone and/or 1,4-dioxan-2-one. The synthetic wound agent also relates to a synthetic wound gel and a cosmetic.

### CROSS-REFERENCE TO RELATED APPLICATIONS

This continuation application claims priority to PCT/EP2022/087354 filed on Dec. 21, 2022, which has published as WO 2023/118370 A1, and also the German application number DE 10 2021 215 013.6 filed on Dec. 23, 2021, the entire contents of which are fully incorporated herein with these references.

### DESCRIPTION

### Field of the Invention

The invention relates to a wound remedy, a wound gel and a cosmetic, in particular, for skin care.

### Background of the Invention

With larger external tissue defects, there is often a greatly increased risk of infection or a bacterial (mixed) infection of the injured tissue area which is often already present. Such wounds often only heal with pronounced scarring. In clinical practice, the tissue defects often must be covered with a skin substitute in the form of a synthetic or biological surface material in order to support secondary wound healing and counteract infections. However, especially with deep or irregular tissue defects, the effect of such surface materials in supporting wound healing is sometimes quite limited. Even when using vacuum foam systems and granulation-promoting artificial skin coverings in deeper tissue layers, wound healing can be unsatisfactory and delayed, making repeated surgical interventions and wound refreshment necessary.

### SUMMARY OF THE INVENTION

It is therefore the object of the invention to provide a synthetic wound agent and a synthetic wound gel that are suitable for use over large areas and that support wound healing, particularly in the case of deeper tissue defects. In addition, the object of the invention is to provide a synthetic cosmetic with a skin-neutral anti-inflammatory effect that is particularly suitable for the aftercare of healed skin wounds.

The object relating to the wound agent is achieved by a wound agent having the features specified in the independent claim. The wound gel or cosmetic according to the invention has the features specified in other claims. Preferred developments of the invention are specified in the subclaims and the description.

The wound agent according to the invention is in the form of a bulk material comprising resorbable particles with a maximum dimension, i.e., a maximum length, of 5 millimeters. The particles each have a porosity of over 80%, wherein the particles each comprise a co-and/or terpolymer based on polyhydroxycarboxylic acids, namely the monomers lactide, trimethylene carbonate (TMC), caprolactone and/or 1,4-dioxan-2-one or polyhydroxybutyrate (PHB). The particles particularly preferably each comprise a copolymer based on lactide and TMC or based on lactide and caprolactone or a terpolymer with lactide and TMC or with lactide and caprolactone and additionally 1,4-dioxan-2-one or polyhydroxybutyrate (PHB).

Due to its particle structure, the synthetic wound dressing can be introduced as a bulk material into deeper wound areas that are only partially or not accessible for surface materials, for example by blowing them in, and thus exert its wound-healing-supporting effect there. The bulk material is suitable for both larger and smaller tissue defects and can be introduced particularly easily into deeper wound sections. Overall, a tissue defect can be completely filled or filled with the wound dressing. Due to the enormous porosity of the particles, the wound dressing can effectively absorb and adsorb wound secretions, inflammatory exudates and blood. Lactate released during the absorption of the wound dressing makes it possible to keep the pH value in the area of the entire tissue defect, i.e., even at the difficult-to-access wound base, in the (slightly) acidic range, which has a germicidal effect and promotes wound healing overall. Overall, the wound dressing can exert an anti-infective and pain-reducing effect, with complete hydrolytic and enzymatic degradability. Given the complete resorbability of the wound agent, subsequent removal of the wound agent is completely unnecessary. This is also beneficial for the formation of granulation tissue as part of the secondary wound closure. The wound agent can be provided in a sterile form without any problems. According to the invention, the bulk particles can, in particular, have a maximum dimension of 2 mm. This means that the wound agent can also be introduced into fine wounds with only small dimensions.

According to a particularly preferred development of the invention, the bulk particles of the wound agent can be at least partially or entirely powdery, i.e., as microparticles. In this case, the particles have a maximum dimension of 700 μm or 150 μm, very particularly preferably only 100 μm. This means that the powdery wound agent can be introduced into even the smallest wound depressions and can take effect there. The wound agent can be provided in a sterile manner without any problems. In addition, the volume of a wound with such a particle size can be filled without any cavities or essentially without any cavities, i.e., without any large cavities between the particles of the wound agent. As a result, a microenvironment that promotes wound healing can be established in the defect volume of the damaged tissue and unwanted infection can be counteracted. The powdered bulk material can be blown into external wounds or—for adhesion prophylaxis—intraperitoneally, particularly with a maximum particle size of 250 μm or 100 μm.

For larger particles, the maximum dimensions of the bulk material particles can be determined by measuring the individual particles using a light microscope. If the powdered bulk material has particles with a maximum dimension of 250 μm or even just 100 μm, the respective maximum dimensions of the bulk material particles can also be determined experimentally using scanning electron microscopy.

The determination of the respective maximum dimensions of the particles using both light microscopy and scanning electron microscopy can be carried out partially or fully automatically, particularly using computer-based optical analysis software. This is advantageous for the rapid and error-free testing of the size of the particles.

The majority or all of the particles of the bulk material/wound agent preferably have a spiky, i.e., irregular, basic shape. The particles are therefore not spherical or rounded. This means that the wound agent can have a mechanically stabilizing function for the tissue surrounding the wound after application to the wound. This can also further promote wound healing until the wound agent is completely resorbed.

According to the invention, the particles of the wound agent preferably have an average pore size in the range of 0.1 μm to 50 μm, in particular, in the range of between 0.5 μm and 30 μm. The average pore size in conjunction with the extraordinarily high porosity of the particles of over 90% ensures particularly reliable absorption of liquids. The inner surface of the particles can thus be quickly and comprehensively wetted with liquid and, not least for this reason, the particles can be reliably reabsorbed.

The wound treatment agent in the form of the powdery bulk material explained above, i.e., in the form of the micropowder, preferably has a bulk density, i.e., a ratio of the mass of the bulk material to the bulk volume occupied, between 0.08 g/ml and 0.25 g/ml, in particular, between 0.12 and 0.14 g/ml according to the measurement method defined in DIN EN ISO 60:2000-01.

If the particles in the sense of micrografts have a maximum dimension between 1 millimeter and 5 millimeters, the bulk material or wound treatment agent can preferably have a volume weight between 0.03 g/ml and 0.06 g/ml, in particular, of approximately 0.04 g/ml.

According to the invention, the particles of the bulk wound agent preferably have a bimodal pore structure or distribution of their pore size with a first pore size in the range from 0.1 μm to 50 μm, in particular, in the range from 0.5 to 30 μm, and with a second pore size in the range from 80 μm to 600 μm, in particular, in the range from 100 μm to 500 μm. This enables the particles to absorb liquid particularly quickly and effectively when the wound agent is applied to the wound. Such a bimodal pore structure can be achieved on the one hand by the drying process described below of a polymer solution used to produce the particles and by additionally providing the polymer solution with sugar before the start of the drying process and removing the sugar at a later time.

According to the invention, the particles can comprise a polyhydroxycarboxylic acid, in particular, a terpolymer of 65 to 90 wt. % lactide (in particular, D,L-lactide), 5 to 20 wt. % trimethylene carbonate and 5 to 20 wt. % ε-caprolactone. In the terpolymer, the monomers lactide, trimethylene carbonate and ε-caprolactone can be present, in particular, in the range of 85/10/5 to 70/20/10 wt. % or mixtures thereof.

According to the invention, the material of the wound dressing can have a glass transition temperature Tg between 22° C. and 45° C. This means that the wound dressing remains dimensionally stable at the ambient temperatures that are usual during transport and storage and does not show any undesirable agglomerations. This ensures that the wound dressing is free flowing. Only when applied can it soften due to the local heat effect of the surrounding (wound) tissue and thus adhere optimally to the (wound) tissue. The glass transition temperature of the material of the wound dressing is determined in a known manner using dynamic differential thermal analysis in accordance with DIN EN ISO 11357-1.

According to the invention, the particles can each have a free monomer content of 0 to 12 wt. %, in particular, 0.1 to 12 wt. %, preferably 1 to 10 wt. %, based on the weight of the co-and/or terpolymer.

According to a preferred development of the invention, the co- and/or terpolymer can be doped with a filler material, in particular, a synthetic polymer, preferably polyvinyl alcohol (PVA). The emulsifying effect of the PVA simplifies the production of the particles.

The majority or all of the particles of the bulk material/wound agent preferably have a spattered basic shape. This allows the particles of the wound agent to interlock with one another after application to a wound and thus stabilize their position and location relative to the wound surfaces—and thus also the wound surfaces themselves—relative to one another. This is beneficial for wound healing.

The wound gel comprises the wound agent explained above and can be used, in particular, as a wound dressing or for intraperitoneal adhesion prophylaxis in the medical treatment of humans and/or animals. The wound gel can be injected or sprayed into the abdominal cavity for this purpose.

According to the invention, the wound gel can comprise one or more other medicinal substances and/or additives in addition to the wound agent (and water). Suitable additives can be used to adjust, for example, the consistency, shelf life, cooling effect of the wound gel, etc. The wound gel can thus comprise, in particular, a biocompatible thickener such as a water-soluble and/or swelling non-ionic polymer, in particular, a poloxamer, polyvinyl alcohol, polyethylene glycol, hyaluronic acid, collagen, gelatin, alginate or a cellulose derivative such as hydroxypropylmethylcellulose, or an emulsifier, such as polyglyceryl-2 triisostearate, or other carriers such as saline solution or Vaseline.

The wound care agent can also be used as a filler, auxiliary agent or active ingredient in cosmetics, in particular, for skin care.

The wound agent according to the invention can be produced, for example, in the manner described below:

In a first step, a polymer solution is prepared from a co- and/or terpolymer based on the monomers lactide, trimethylene carbonate, caprolactone and/or 1,4-dioxan-2-one or polyhydroxybutyrate and a suitable solvent, for example dimethyl sulfoxide (DMSO). For example, a 10% polymer solution is prepared with 4% PVA (polyvinyl alcohol) in dimethyl sulfoxide (DMSO).

The polymer solution is then applied to a, preferably flat, carrier. This can be done, for example, by draining the polymer solution, for example through a sieve with a defined mesh size of, for example, 0.042 mm. The carrier can be a glass plate, for example. The polymer solution can be drawn out onto the carrier into a film using a doctor blade or a film-drawing device and can also be provided with an excess of sugar, preferably sucrose.

The solvent is removed in a subsequent step by drying, in particular, by freeze-drying, to form a polymer cake.

Drying can be done as follows:

For freeze-drying, the polymer solution is preferably kept at −60° C. and a vacuum (i.e., at a subatmospheric ambient pressure) of ≤0.5 mbar for at least 12 hours in the first sub-step.

If necessary, the polymer cake can subsequently be incubated in a bath filled with deionized water (fully demineralized water) with circulation and temperature control (at ˜21° C.) for a period of approx. 40 min-70 min and then easily removed from the carrier.

The polymer cake is then placed in bidistilled water and washed for 2 hours each and then freeze-dried for at least 12 hours at a vacuum of ≤0.5 mbar and −60° C.

In a subsequent optional step, the polymer cake 58 is dried under clean room conditions at approximately +21° C. and 30% relative humidity for at least another 12 hours.

The polymer cake thus obtained is then comminuted, in particular, by single- or multi-stage crushing or grinding or by other known methods, to form a bulk material or wound dressing with the respective predetermined maximum particle dimension d.

For example, the polymer cake can be pre-crushed into approximately 2×2 cm polymer cake pieces and preferably cooled with liquid nitrogen. The liquid nitrogen can pre-embrittle the polymer cake and thus improve processability. These polymer cake pieces can then be further crushed into wound dressing10/bulk material12 using a cryomill (e.g. with a 12-tooth rotor at 18,000 rpm and with a ring sieve 1.0-0.5 mm) together with a sufficient amount of liquid nitrogen for cooling/embrittlement. The cryomill used can be the ultra-centrifugal mill ZM 200 from RETSCH GmbH, Retsch-Allee 1-5, 42781 Haan, Germany. Any particles that may still be too large in the bulk material can, if necessary, be detected and removed from the bulk material by optional pre-sieving of the bulk material (according to DIN 66165) and then based on an optical (light microscopic or scanning electron microscopic) size determination of the particles of the bulk material.

The bulk material/wound agent obtained in this way is then preferably vacuum dried for at least 12 hours at an atmospheric pressure of less than 2 mbar. This allows the bulk material to be provided with a degree of dryness suitable for packaging, storage and transport purposes and thus ensures the flowability of the wound agent when it is applied.

Furthermore, the bulk material according to the invention can be used as a matrix for a nutrient medium for cultivating animal or plant cells, i.e., for cell cultures.

The invention is explained below using embodiments shown in the drawing. The embodiments shown and described are not to be understood as an exhaustive list, but rather have an exemplary character for the description of the invention.

### BRIEF DESCRIPTION OF THE DRAWINGS

In the drawing show:

FIG. 1A is an overview SEM image of a wound treatment agent in the form of debris made of resorbable particles, here with an example of a maximum particle size between 500 μm and 1 mm;

FIG. 1B is a detailed section SEM image of a wound treatment agent in the form of debris made of resorbable particles, here with an example of a maximum particle size between 500 μm and 1 mm in a detailed section (FIG. 1B);

FIG. 2A is an overview SEM image of a wound care product in the form of bulk material made of resorbable particles with a maximum particle size between 250 μm and 500 μm;

FIG. 2B is a detailed section SEM image of a wound care product in the form of bulk material made of resorbable particles with a maximum particle size between 250 μm and 500 μm;

FIG. 3A is an overview SEM image of a wound care product in the form of bulk material made of resorbable particles with a maximum particle size between 63 μm and 250 μm;

FIG. 3B is a detailed section SEM image of a wound care product in the form of bulk material made of resorbable particles with a maximum particle size between 63 μm and 250 μm;

FIG. 4A is an overview SEM image of a single particle of a wound healing agent in the form of bulk material;

FIG. 4B is a highly magnified detail SEM image of a single particle of a wound healing agent in the form of bulk material;

FIG. 5 is a SEM image of a particle of a wound-treating agent similar to the particle shown in FIG. 4, showing the flat or essentially flat underside;

FIG. 6 is a block diagram with individual process steps of a manufacturing process for a wound treatment agent in the form of bulk material; and

FIG. 7 is a schematic representation of a wound gel or a cosmetic.

### DETAILED DESCRIPTION OF EMBODIMENTS OF THE INVENTION

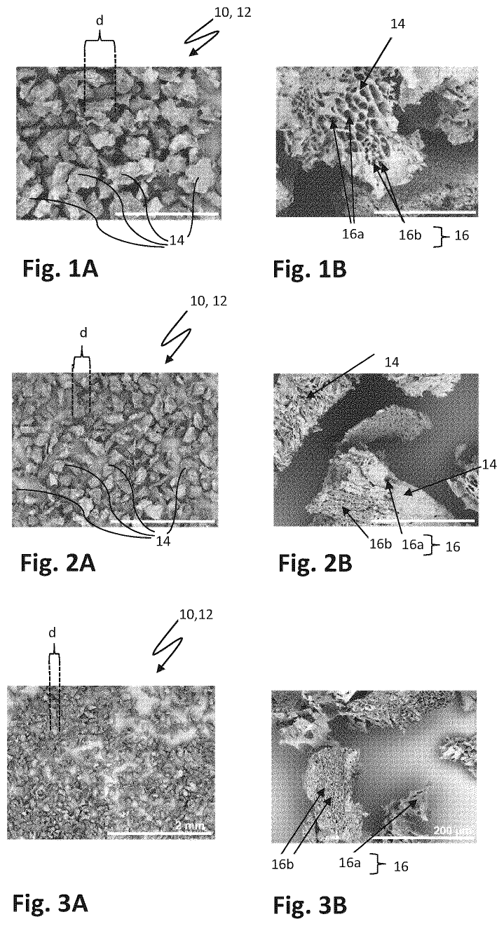

FIGS. 1 to 3 each show scanning electron microscope (SEM) images of wound care products 10. The scale shown in FIGS. 1A, 2A, 3A and not specified in detail corresponds uniformly to a length of 2 mm and in FIGS. 1B, 2B, 3B to a length of 200 μm.

The wound agents 10 shown are each in the form of bulk material 12, which consists of a large number of particles 14. Thus, the wound agent 12 according to FIG. 1 has particles 14 with a maximum dimension d (=maximum length) of between 500 μm and 1 mm. The wound agent 12 shown in FIG. 2 has particles with a maximum dimension of between 250 μm and 500 μm, and the wound agent 12 shown in FIG. 3 has particles 14 with a maximum dimension of between 63 μm and 250 μm. The bulk wound agent 12 according to FIGS. 1A, 1B, 2A and 2B has a volume weight of between 0.08 g/ml and 0.16 g/ml, in particular, between 0.12 and 0.14 g/ml.

The porous structure of the particles 14 is clearly visible. The porosity of the particles 14 is over 90%. The pores 16 can be differentiated into larger primary or macropores 16a and smaller secondary or micropores 16b. The majority or all of the particles 14 have a spiky, i.e., irregular, basic shape. This allows the particles to get caught in one another in the wound. In this way, the wound edges of a wound can be mechanically stabilized relative to one another by the wound powder introduced into the wound.

The different degrees of fineness of the bulk wound agent 10 allow a particularly wide range of applications for the wound agent 10. The finer, i.e., smaller the particle size, the better the wound agent 10 can be used for smaller and smallest wounds.

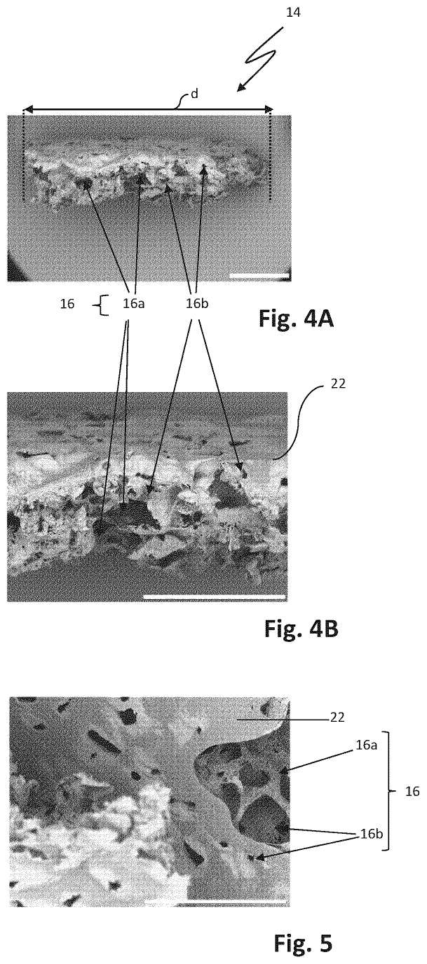

FIGS. 4A and 4B show SEM images of a single particle 14 of a wound agent 10 at different magnifications. The particle 14 here has a maximum dimension d of 3 millimeters. The particle 14 can therefore be referred to as a micrograft and is ideal for treating large and irregularly defined wounds. The porous structure of the particle 14 can be clearly seen in FIG. 4B. Here, too, the pores 16 can be differentiated into larger primary or macropores 16a and smaller secondary or micropores 16b. The particle 14 also has a spattered basic shape with irregularly defined side flanks 18. The spattered basic shape of the particle 14 is due to the mechanical comminution of a larger polymer cake during the production of the bulk material or the particle 14. The underside 22 of the particle 14 shows a predominantly closed and slightly wavy topography with individual pores 16b. According to the SEM image of another particle 14 shown in FIG. 5, the underside 22 can be relatively flat and have only a few macropores 16a and numerous micropores 16b. The scale shown in FIGS. 4 and 5 corresponds to 1 mm in each case.

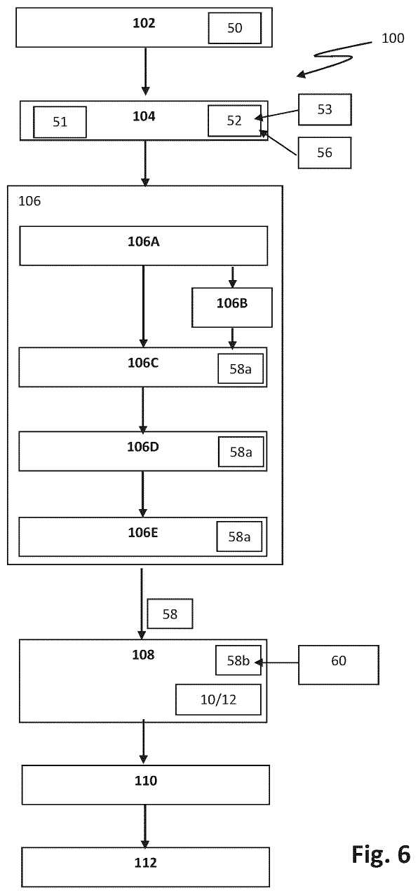

FIG. 6 shows a block diagram with individual method steps of a manufacturing method 100 for a wound agent 10 explained above in connection with FIGS. 1 to 5.

In a first step 102, a polymer solution 50 is prepared from a (of course previously provided) co-and/or terpolymer based on the monomers lactide, trimethylene carbonate, caprolactone and/or 1,4-dioxan-2-one or polyhydroxybutyrate (PHB) and a suitable solvent. For example, a 10% polymer solution 50 can be prepared with 4% PVA (polyvinyl alcohol) in DMSO.

This polymer solution 50 is applied to a carrier 52 in step 104, for example while draining the polymer solution (for example via a sieve 51 with a mesh size of 0.042 mm). The carrier 52 can, in particular, be a glass plate. The polymer solution 50 can be drawn out to form a film on the carrier 52 by means of a doctor blade 54 or a film drawing device and can be provided with an excess of sugar 56 in order to form the macropores 16a explained above.

In a further step 106, a polymer cake 58 is produced by removing the solvent by drying, in particular, by freeze-drying.

The polymer solution 50 is passed through a sub-step 106A Freeze-dried for at least 12 hours at −60° C. and a vacuum of ≤0.5 mbar.

The polymer pre-cake 58a produced in this way can be incubated in the optional intermediate step 106B in a bath filled with deionized water (fully demineralized water) with circulation and temperature control (˜21° C.) for approx. 40 min-70 min and then easily detached from the carrier.

The polymer pre-cake 58a is placed in bidistilled water in the subsequent step 106C and washed for 2 hours each and then freeze-dried in step 106D for at least 12 hours at least −60° Celsius and a vacuum of ≤0.5 mbar.

In the following step 106E, the polymer pre-cake 58a can be dried under clean room conditions at approximately +21° C. and 30% relative humidity for at least another 12 hours.

The polymer cake 58 thus obtained is mechanically comminuted in a subsequent step 108 to form the wound treatment agent 10 in the form of bulk material 12 with the respectively specified maximum particle size/dimension d (cf. FIGS. 1-4) of the particles 14. This can be done, in particular, by single or multi-stage crushing or grinding or by other techniques corresponding to the state of the art.

Pre-comminuted, in particular, to approximately 2×2 cm polymer cake pieces 58b and cooled with liquid nitrogen 60. These polymer cake pieces 58a can then be further comminuted to form wound dressing 10/bulk material 12 together with a sufficient amount of liquid nitrogen for cooling using a cryomill (e.g., with a 12-tooth rotor at 18,000 rpm and with a ring sieve 1.0-0.5 mm).

The thus obtained (powdered) wound agent 10/bulk material 12 is preferably vacuum dried in step 110 for at least 12 hours at an atmospheric ambient pressure of less than 2 mbar.

Any particles that may still be too large in the wound agent 10/bulk material 12 can, if necessary, be removed from the wound agent 10/bulk material 12 before or after step 110 by optional pre-sieving of the bulk material (according to DIN 66165) and on the basis of an optical (light microscopic or scanning electron microscopic) size determination 112 of the particles of the wound agent 10/bulk material 12.

The mechanical comminution preferably takes place at a low air humidity or a humidity of the ambient atmosphere of less than 40%, in particular, less than 20%. In the same way, the wound agent 10 is preferably vacuum-packed under the same conditions and thus made available for use on humans/animals or for further processing.

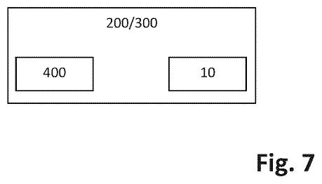

FIG. 7, the wound agent 10 can also be used as a component of a wound gel 200. In other words, the wound gel 200 comprises the (preferably micro-) particulate wound agent 10. The wound gel 200 can also contain water and a water-soluble non-ionic polymer 400, in particular, a cellulose derivative such as hydroxypropylmethylcellulose, or a polyglyceryl-2 triisostearate as an emulsifier, as well as film formers. The wound gel is particularly suitable for intracorporeal, in particular, intra-abdominal application, for example for adhesion prophylaxis after abdominal interventions. The wound gel 200 can be conveniently applied, in particular, injected, in the surgical site even with minimally invasive surgical access. If required, the wound gel 200 can also contain a dye, which can be used to visually check the surface application in a simplified manner.

According to FIG. 7, the wound treatment agent 10 can also be used as a filler and active ingredient in cosmetics 300, in particular, those for external use. Here, the wound treatment agent 10 can prevent skin irritations and micro-inflammations of the skin as a precursor to acne and promote faster regeneration of the skin.

In addition, the bulk material 12 explained above can also serve as a carrier system for drugs. Due to its biocompatibility and complete resorbability, the carrier system can enable a slower release of the drug and thus a long-term effect of the drug. It is advantageous that the bulk material 12 is not toxic in either extracorporeal or endocorporeal surface application and can easily be provided in a sterile manner.

What is claimed is:

1. A wound agent in the form of bulk material comprising particles with a maximum dimension (d) of 5 mm, wherein the particles each have a porosity of more than 80% and each comprise a co- and/or terpolymer based on polyhydroxycarboxylic acids, the monomers comprising lactide, trimethylene carbonate, caprolactone and/or 1,4-dioxan-2-one or polyhydroxybutyrate (PHB).

2. The wound agent according to claim 1, wherein the particles at least partially or all have the maximum dimension (d) of 2 mm.

3. The wound agent according to claim 1, wherein the particles at least partially or all have the maximum dimension (d) of 700 μm or 150 μm.

4. The wound agent according to claim 1, wherein the particles have an average pore size in the range of 0.1 μm to 50 μm.

5. The wound agent according to claim 1, wherein the particles have an average pore size in the range of 0.5 to 30 μm.

6. The wound agent according to claim 2, wherein the particles have a bulk density between 0.08 g/ml and 0.16 g/ml.

7. The wound agent according to claim 2, wherein the particles have a bulk density between 0.12 and 0.14 g/ml.

8. The wound agent according to claim 1, wherein the particles have a maximum dimension (d) between 1 mm and 5 mm and a volume weight between 0.03 g/ml and 0.06 g/ml.

9. The wound agent according to claim 1, wherein the particles have a maximum dimension (d) between 1 mm and 5 mm and a volume weight of approximately 0.04 g/ml.

10. The wound agent according to claim 1, wherein the particles have a bimodal pore structure with a pore size in the range from 0.1 μm to 50 μm, and in the range from 80 μm to 600 μm.

11. The wound agent according to claim 1, wherein the particles have a bimodal pore structure with a pore size in the range from 0.5 to 30 μm, and in the range from 100 μm to 500 μm.

12. The wound agent according to claim 1, wherein the particles each have a content of free monomers of 0 to 12 wt. %, in particular, 0.1 to 10 wt. %, preferably 1 to 9 wt. %, based on the weight of the co- and/or terpolymer.

13. The wound agent according to claim 1, wherein the particles each have a content of free monomers of 0.1 to 10 wt. % based on the weight of the co- and/or terpolymer.

14. The wound agent according to claim 1, wherein the particles each have a content of free monomers of 1 to 9 wt. % based on the weight of the co- and/or terpolymer.

15. The wound agent according to claim 1, wherein the co- and/or terpolymer is doped with a filler material, being a synthetic polymer.

16. The wound agent according to claim 1, wherein the co- and/or terpolymer is doped with a filler material, being polyvinyl alcohol (PVA).

17. The wound agent according to claim 1, wherein the majority or all of the particles have a sprinkled basic shape.

18. A wound gel or a wound cosmetic, wherein the wound gel or the wound cosmetic comprises the wound agent according to claim 1.

19. The wound gel or the wound cosmetic according to claim 18, wherein the wound gel or the wound cosmetic comprises a water-soluble non-ionic polymer, being a cellulose derivative.

20. The wound gel or the wound cosmetic according to claim 18, wherein the wound gel or the wound cosmetic comprises a water-soluble non-ionic polymer, being a hydroxypropylmethylcellulose.

21. The wound gel or the wound cosmetic according to claim 18, wherein the wound gel or the wound cosmetic comprises a water-soluble non-ionic polymer, being a polyglyceryl-2 triisostearate.
